(19) United States
(12) Patent Application Publication
(10) Pub. No.: US 2011/0183013 A1
(43) Pub. Date: Jul. 28, 2011

(54) Herbal composition for improving skin quality and method for making the same

(76) Inventor: Yen-Tzu Wang, Zhonghe City (TW)

(21) Appl. No.: 12/656,330

(22) Filed: Jan. 26, 2010

(51) Int. Cl. A61K 8/23; A61Q 19/08

(52) U.S. Cl. 424/705

(57) ABSTRACT

An herbal composition for improving skin quality and a method for making the herbal composition are proposed. The herbal composition is obtained by mixing vinegar and a small amount of sulfur into a primary composition, adding a secondary composition for soaking, and then filtering residues. The method includes steps of: (a) adding vinegar into a soaking container; (b) adding a small amount of sulfur into the soaking container; (c) adding a secondary composition into the soaking container; and (d) filtering residues of those added into and soaked in the soaking container previously so as to obtain the herbal composition.

### FIELD OF THE INVENTION

The present invention relates to an herbal composition for improving skin quality and a method for making such composition, wherein the herbal composition is obtained by mixing vinegar and a small amount of sulfur becomes a primary composition, adding a secondary composition for soaking, and then filtering residues. The resultant herbal composition is advantageous for having the function of improving skin quality, being pharmacologically mild and brining no side effect. The herbal composition may be further manufactured into membranes or cream.

### BACKGROUND OF THE INVENTION

It is known that aging signs such as wrinkles and age spots appear on human skin with not only age but also external stimulus, like sunshine or low-moisture environments. This makes people who appreciate beauty or pay attention to skin care seek for approaches to improving skin quality by retarding or remedying these aging signs.

While modern western medicine has proposed many popular solutions for improving human skin quality, such as Glycolic Peeling and the like, it is to be noted that these solutions and the means they implement may bring additional irritation to our skin, more or less. Especially, when it comes to people allergic to some particular chemical or pharmaceutical ingredients, side effects such as allergic reaction, tumidity, irritation or skin injury may happen. Hence, there is a pressing need to develop a pharmacologically mild herbal composition for improving skin quality.

Vinegar is presented in various types, including fermented vinegar, industrial vinegar and food vinegar, all being sour and some being applicable for medical purposes. Vinegar is a strong disinfectant and is helpful to protection of skin and hairs.

Sulfur has been long used in Chinese medical science as a mineral drug, with the recognized effects on curing impotence and sterilization. For sterilization, sulfur is typically used in the form of external skin cures. Many people enjoy sulfur-containing spring not only because it treats skin diseases, but also because it has the functions of softening skin cuticle, relieving itching, and detoxifying. Sulfur-containing spring is effective in mitigating some chronic skin diseases, such as skin pruritus, keratosis, chronic eczema, skin mycosis, and pyoderma, and is also constructive in easing pain and itch.

### SUMMARY OF THE INVENTION

In view of the need of a pharmacologically mild herbal composition for improving skin quality, one objective of the present invention is to provide an herbal composition for improving skin quality, which is effective in improving skin quality, pharmacologically mild, and incurs no side effect.

Another objective of the present invention is to provide a method for making an herbal composition for improving skin quality, wherein the herbal composition is obtained by mixing vinegar and a small amount of sulfur becomes a primary composition, adding a secondary composition for soaking, and then filtering residues. The obtained herbal composition may be further manufactured into membranes or cream.

To achieve these objectives, the present invention provides an herbal composition for improving skin quality, wherein the herbal composition is obtained by mixing vinegar and a small amount of sulfur becomes a primary composition, adding a secondary composition for soaking, and then filtering residues. The herbal composition is able to effectively improving skin quality, and is pharmacologically mild while brining no side effect.

The present invention also provides a method for making an herbal composition for improving skin quality. The method includes steps of: (a) adding vinegar into a soaking container; (b) adding a small amount of sulfur into the soaking container for mixing the sulfur with the vinegar; (c) adding a secondary composition into the soaking container for soaking; and (d) filtering residues of those added into and soaked in the soaking container previously so as to obtain the herbal composition, which is effective in improving skin quality and pharmacologically mild while brining no side effect.

### BRIEF DESCRIPTION OF THE DRAWINGS

The invention as well as a preferred mode of use and advantages thereof will be best understood by referring to the following detailed description of the illustrative embodiments in conjunction with the accompanying drawings, wherein:

FIG. 1 is a flowchart according to a first embodiment of the present invention; and

FIG. 2 is a flowchart according to a second embodiment of the present invention.

### DETAILED DESCRIPTION OF THE INVENTION

The present invention provides an herbal composition for improving skin quality and a method for making the herbal composition, wherein the herbal composition is effective in improving skin quality and brings no side effect, namely being mild. Of course, the herbal composition may be manufactured membranes or cream.

The herbal composition of the present invention is primarily made by mixing vinegar and a small amount of sulfur as a primary composition, adding secondary composition in the primary composition for soaking, and filtering the residues from the herbal composition. In one preferred embodiment, the primary composition is composed of vinegar, a small amount of sulfur and glacial acetic acid. In another preferred embodiment, in addition to the primary composition, a secondary composition, a primary additive that has stewed in water and a secondary additive that has stewed in water are further included in the herbal composition, wherein the secondary composition comprises: Plantago Asiatica, Cnidii Fructus, Kochiae Fructus, a small amount of Pricklyash Peel or combination thereof, and the primary additive comprises: Purslane, Artemisia Argyi, Aloe or combination thereof while the secondary additive comprise: Rhei Rhizoma, Phellodendri Cortex, Rhizoma Atractylodis, Baikalskullcap Root, Citronella, Formosan Torenia or combination thereof. The vinegar may be fermented vinegar, industrial vinegar or food vinegar.

Plantago Asiatica contains rich carbohydrate, including Plantasan, L-Rhamnose, D-Galactose, etc. Plantago Asiatica oozes mucilage when immersed in water. Psyllium is an herbal plant belonging to Ginuceae family and is commonly used in resisting skin irritative reaction. Cnidii Fructus is typically used externally for curing scabies and eczema. Kochiae Fructus refers to dried, matured seeds of Kochia scoparia (L.) Schrad. [Chenopodium scoparium L.]. Its water extract provides different levels of inhibition to common pathogenic skin fungus, such as Achorion Schonleinii, M audouinii, M Ferruginum, Microsporum, and Nocardia. Pricklyash Peel belongs to Zanthoxylum family, and is also known as zanthoxylum and Chinese peppe. Its seeds are effevitive in sterilization, disinfection, pain relief, itch relief, and detumescence, and is efficacious to suppress various bacteria, especially bacteria existing on skin surface. Therefore, it is clinically used in treating skin diseases such as eczema, pruritus and neurodermatitis.

Phellodendri Cortex, also known as Bark of Chinese Corktree, and Amur Corktree Bark, kills germs. Rhizoma Atractylodis is efficacious in curing colds, removing internal damp, treating epidemic pathogenic factors from mountains, and remedying edematous skin. Radix Scutellariae whose scientific name is Scutellaria baicalensis Georgi, has a wide antibacterial spectrum, and helps to cure carcinomatous dermatitis and atopic dermatitis, and Radix Scutellariae is good at suppressing Corynebacterium Acnes. The scientific name of Purslane is Portulaca oloraua Linn. Var. sativa DC. Purslane contains rich flavone, epinephrine, polysaccharides, and various compounds such as vitamins and amino acids. The extract of Purslane has the effects of treating skin allergies, resisting external stimulative factors to skin, remedying inflammatory and treating acnes. When used externally, Purslane also helps to improving furuncles and purulent diseases, as well as eczema, lacquer dermatitis, dermatitis, skin pruritus and pain. The scientific name of Formosan Torenia is Torenia concolor Lindley var. Formosana Yamazaki, and the whole plant is useful in reducing inflammation, relieving fever, and treating sunstroke, dysentery, colds, as well as bone ache. Artemisia argyi serves to clean, sterilize and is astringent. It provides the functions of balancing Oil excretion, calming skin, and reducing inflammation. When used before juniper lotion, Artemisia argyi can facilitate prompting skin absorption, keeping skin fresh, maintaining desired skin moisture and repairing skin texture, thereby cause skin tender and shining. Aloe has four major features. The first is permeability. Since Aloe extract has a pH value similar to that of human skin, contents of Aloe, such as vitamins and mineral substances can easily permeate into and thus get absorbed by skin. The second feature is aging resistance. Aloe contains a lot of saponin that helps to improve collagen synthesis and thus resist skin aging. The third feature is whitening skin. Aloe contains aloin that prevents human melanin generation and helps to fading skin spots, thereby whitening human skin. The last feature is its water-retaining ability. Aloe also has a lot of polysaccharides, which helps to retain moisture in human skin, thus keeping skin tender. Citronella is also sknoe as citronella Cymbopogon citratus (DC.) Stapf. belonging to citronella category in the grass family. Its whole plaint can be sued as drug and it is particularly effective in improving enlarged pores while also working in treating skin fungal infections, such as Athelete's Foot. Rhei Rhizoma is famous for its functions for relieving internal fever and improving skin texture revivification.

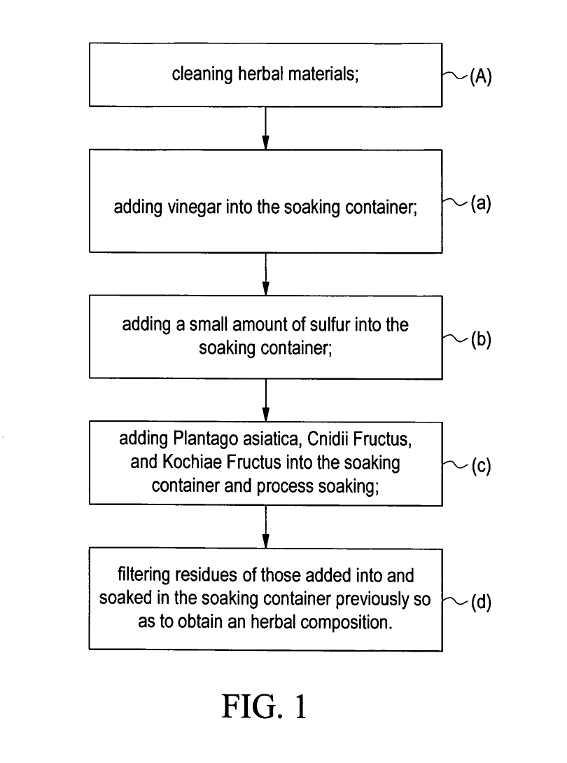

FIG. 1 illustrates a first embodiment of the method of the present invention, wherein the following steps are included:

(A) Cleaning Herbal Materials;

(a) adding vinegar (the primary composition) into a soaking container, wherein the vinegar may be fermented vinegar, industrial vinegar or food vinegar;

(b) adding a small amount of sulfur (the primary composition) into the soaking container;

(c) adding Plantago asiatica, Cnidii Fructus and Kochiae Fructus (all belonging to the secondary composition) into the soaking container and process soaking; and

(d) filtering residues of those added into and soaked in the soaking container previously so as to obtain an herbal composition.

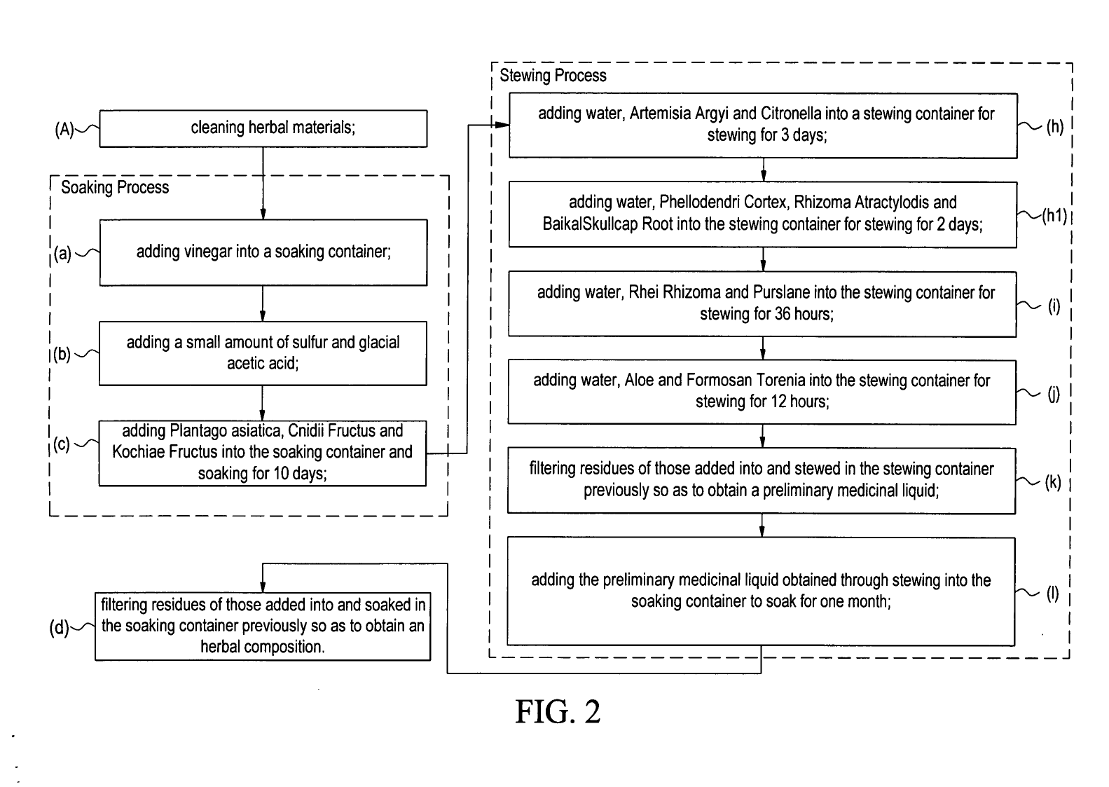

Please refer to FIG. 2 for a method according to a second embodiment of the present invention. It comprises the following steps:

(A) Cleaning Herbal Materials;

followed by soaking process:

(a) adding vinegar (the primary composition) into a soaking container, wherein the vinegar may be fermented vinegar, industrial vinegar or food vinegar;

(b) adding a small amount of sulfur and glacial acetic acid (both are the primary composition) into the soaking container;

(c) adding Plantago asiatica, Cnidii Fructus and Kochiae Fructus (all belonging to the secondary composition) into the soaking container and soaking for 10 days;

(B) Stewing Process:

(h) adding water, Artemisia Argyi (the primary additive) and Citronella (the secondary additive) into a stewing container for stewing for 3 days (on temperature 100° C. or higher);

(h1) adding water, Phellodendri Cortex, Rhizoma Atractylodis and BaikalSkullcap Root (the secondary additive) into the stewing container for stewing for 2 days (on temperature 100° C. or higher);

(i) adding water, Rhei Rhizoma (the secondary additive) and Purslane (the primary additive) into the stewing container for stewing for 36 hours (on temperature 100° C. or higher);

(j) adding water, Aloe (the primary additive) and Formosan Torenia (the secondary additive) into the stewing container for stewing for 12 hours (100° C. or higher);

(k) filtering residues of those added into and stewed in the stewing container previously so as to obtain a preliminary medicinal liquid;

(l) adding the preliminary medicinal liquid obtained through stewing into the soaking container to soak for one month;

wherein the 3 days in the step (h) plus 2 days in the step (h1) plus 36 hours of the step (i) and 12 hour of the step (j) total compose a 7-day stewing period.

Soaking the preliminary medicinal liquid in the soaking container for one month, so as to make a solution of the primary and secondary compositions mix and soak with the preliminary medicinal liquid of the primary and secondary additives; and

finally process step (d) filtering residues of those added into and soaked in the soaking container previously so as to obtain an herbal composition, which may be further manufactured into membranes or cream.

In other words, the method according to the second embodiment of the present invention includes the steps (a)˜(c), followed by the steps (h)˜(l), and then process final step (d) successively.

Therein, since the soaking period in the soaking process is 10 days, and the stewing period of the stewing process is 7 days, there is a time difference of 3 days between the processes. Therefore, in practice, it is preferred that the stewing period of the stewing process begins at the third day of the soaking period of the soaking process. In other words, the fourth day of the soaking period and the first day of the stewing period are at the same day, and consequently, the tenth day of the soaking period is just the seventh day of the stewing period so that the first soaking period and the stewing period are finished at the same time. Afterward, the stewed preliminary medicinal liquid can be added into the soaking container that has undergone the 10-day soaking process. After that, the one month soaking period is started.

In addition, the rate between vinegar and the herbal composition is preferably 1:10˜1:20 (while 1:10 is more preferred).

For example, vinegar is 22 kg, and the herbal composition is 220 kg.

The rate between glacial acetic acid and the herbal composition is preferably 1:35˜1:50 (while 1:37 is more preferred).

For example, glacial acetic acid is 30 kg, and the herbal composition is 1,110 kg.

The present invention has the following features. Since the present invention relates to an herbal composition, the product is pharmacologically mild. The novel formula and processed of the present invention endows the herbal composition with effectiveness of improving skin quality and bringing no side effect. The disclosed herbal composition thus can renew skin in a mild way and brings no side effect. While renewing skin, the herbal composition brings the effects of the herbs into full play to repairing skin, so as to improve skin quality. In virtue of the compositions and manufacturing method of the present invention, the produced herbal composition is effective in improving human skin quality and may be further manufactured into membranes or cream. That is to say, the present invention has exactly overcome the limit of conventional skin treatment related to the western medicine that can sometime cause side effects such as allergies, tumidity, irritation, and even injuries to human skin, and provides people with a herbal composition, or further manufactured into membranes or cream that helps to improve skin quality and is pharmacologically mild while bringing no side effect.

The embodiments described above are intended only to demonstrate the technical concept and features of the present invention so as to enable a person skilled in the art to understand and implement the contents disclosed herein. It is understood that the disclosed embodiments are not to limit the scope of the present invention. Therefore, all equivalent changes or modifications based on the concept of the present invention should be encompassed by the appended claims.

What is claimed is:

1. An herbal composition for improving skin quality, the herbal composition being obtained by mixing vinegar and a small amount of sulfur into a primary composition, adding a secondary composition for soaking, and then filtering residues.

2. The herbal composition of claim 1, wherein the primary composition further comprises glacial acetic acid and is made by mixing the glacial acetic acid, vinegar and the small amount of sulfur.

3. The herbal composition of claim 1, wherein the secondary composition comprises Plantago Asiatica, Cnidii Fructus, Kochiae Fructus, a small amount of pricklyash Peel or combination thereof.

4. The herbal composition of claim 1, wherein the herbal composition further comprises a primary additive that has been stewed with water and the herbal composition is made by soaking the primary additive and the secondary composition in the primary composition and filtering residues from the primary composition, the primary additive comprising Purslane, Artemisia Argyi, Aloe or combination thereof.

5. The herbal composition of claim 4, wherein the herbal material further comprises a secondary additive that has been stewed with water and the herbal composition is made by soaking the secondary additive, the primary additive stewed with water and the secondary composition in the primary composition and filtering residues from the primary composition, the secondary additive comprising Rhei Rhizoma, Phellodendri Cortex, Rhizoma Atractylodis, Baikalskullcap Root, Citronella, Formosan Torenia or combination thereof.

6. A method for making an herbal composition for improving skin quality, the method comprising:
(a) adding vinegar into a soaking container;
(b) adding a small amount of sulfur into the soaking container;
(c) adding a secondary composition into the soaking container for soaking; and
(d) filtering residues of those added into and soaked in the soaking container previously so as to obtain an herbal composition.

7. The method of claim 6, further comprising adding secondary composition in the soaking container, the secondary composition comprising Plantago Asiatica, Cnidii Fructus, Kochiae Fructus, Pricklyash Peel, or combination thereof.

8. The method of claim 6, further comprising adding glacial acetic acid into the soaking container for soaking.

9. The method of claim 6, further comprising following steps between the steps
(c) and (d):
(h) adding water and Artemisia Argyi into a stewing container for stewing;
(i) adding water and Purslane into the stewing container for stewing;
(j) adding water and Aloe into the stewing container for stewing;
(k) filtering residues of those added into and stewed in the stewing container previously so as to obtain a preliminary medicinal liquid; and
(l) adding the preliminary medicinal liquid obtained through stewing into the soaking container where the soaking has been carried for a predetermined time period.

10. The method of claim 9, further comprising a step (h1) between the steps (h) and (i), the step (h1) comprising adding Phellodendri Cortex, Rhizoma Atractylodis and BaikalSkullcap Root into the stewing container, adding Citronella into the stewing container in the step (h), adding Rhei Rhizoma into the stewing container in the step (i), and adding Formosan Torenia into the stewing container in the step (j).
